(19) United States
(12) Patent Application Publication
(10) Pub. No.: US 2021/0346403 A1
(43) Pub. Date: Nov. 11, 2021

(54) USE OF COMPOUNDS THAT TARGET MINERALOCORTICOID RECEPTORS FOR TREATING COVID-19

(71) Applicant: Richard HENRY, Glenburnie (CA)

(72) Inventor: Richard HENRY, Glenburnie (CA)

(21) Appl. No.: 17/314,727

(22) Filed: May 7, 2021

Related U.S. Application Data
(60) Provisional application No. 63/022,092, filed on May 8, 2020.

(51) Int. Cl. A61K 31/585

(52) U.S. Cl. CPC A61K 31/585

(57) ABSTRACT

The present disclosure provides the use of a compound that targets mineralocorticoid receptors to treat coronavirus disease 2019 (COVID-19). The compound is preferably one of eplerenone and spironolactone, which are mineralocorticoid receptor antagonists. On one hand, eplerenone may be administered in a range between 25-100 mg twice per day, while spironolactone is administered in a range between 25-100 mg per day. The present disclosure also provides for a method of treatment of COVID-19 using the eplerenone and spironolactone compounds.

### CROSS REFERENCE TO RELATED APPLICATION

The present application claims priority to U.S. Provisional Application No. 63/022,092, entitled “USE OF COMPOUNDS THAT TARGET MINERALOCORTICOID RECEPTORS FOR TREATING COVID-19” filed on May 8, 2020, the contents of which are incorporated herein by reference in their entirety.

### FIELD

The present disclosure relates to the field of treatment for the novel Coronavirus Disease 2019 (COVID-19), a pathological response evoked in certain patients as a result of infection with the novel coronavirus SARS-CoV-2 that is responsible for a pandemic outbreak in 2020.

### BACKGROUND

SARS-CoV-2 is a novel zoonotic coronavirus that, since it emerged in Wuhan, China in December 2019, has spread rapidly around the world. SARS-CoV-2 is more virulent than its predecessor SARS-CoV-1 that caused the SARS outbreak in 2003. This increase in virulence is explained by its gain-of-function genetic mutations to the receptor binding protein domain located on the envelope Spike protein that increases its affinity to the target protein required for cell entry.

Both novel coronaviruses target human angiotensin converting enzyme 2 (ACE2), a membrane protease protein expressed by a variety of cells that respond to the renin-angiotensin system (RAS), a highly conserved system tasked with maintaining interstitial fluid homeostasis by exerting actions to effect salt and water retention in the kidney and increased blood pressure.

ACE2 is a membrane protease co-expressed with angiotensin converting enzyme (ACE). ACE cleaves the pro-protein signal molecule angiotensin-1 (AngI) to the active form, angiotensin-2 (AngII), which exerts its overall effect by binding to target cells expressing angiotensin receptors 1 and 2 (AR₁ & AR₂).

ACE2 essentially exerts a counteractive protective effect on RAS-targeted cells by cleaving both AngI and AngII to form Ang (1-9) and Ang (1-7) respectively, diverting Ang II from AT₁Rs to target the anti-stress Mas receptor instead. In this simplified way, ACE2 both down-regulates Ang II stress and converts it into an anti-stress effect.

COVID-19 is thought to involve widespread viral-induced depletion of ACE2, thereby increasing the target cells to heightened AngII-mediated stress. Treatments to block AngII receptors have not resulted in better outcomes in either SARS or COVID-19 patients.

Current predictions of a worldwide 60% infection rate combined with a current 5% mortality rate indicate that SARS-CoV-2 will cause widespread morbidity and mortality, making it imperative that we find ways to both contain viral spread and treat those infected.

### SUMMARY

In one aspect of this invention patients infected with SARS-CoV-2, or similar ACE2-depleting viruses, will be treated with aldosterone-reducing treatments:

medications/molecules to reduce or prevent the production of aldosterone.

In one embodiment the patient experiencing severe symptoms of COVID-19 will be treated with an appropriate dose of eplerenone and/or spironolactone until the symptoms abate.

In another embodiment, aldosterone-reducing treatment will be undertaken to reduce and prevent fibrosis in both the heart and lungs of patients who survive Covid-19.

In an aspect, the present disclosure provides for a use of a compound that targets mineralocorticoid receptors to treat coronavirus disease 2019 (COVID-19).

In another aspect, the present disclosure provides for a method for treating a patient with a compound that targets mineralocorticoid receptors, wherein the patient is suffering from coronavirus disease 2019 (COVID-19), the method comprising the steps of: determining whether the patient has the COVID-19 by obtaining a biological sample from the patient; and, if the patient has the COVID-19, administering the compound.

In yet another aspect, the present disclosure provides for a use of a compound to prevent post-coronavirus 2019 (COVID-19) heart and lung fibrosis.

In yet another aspect, the present disclosure provides a method for treating a patient with a compound, wherein the patient is suffering from coronavirus disease 2019 (COVID-19), the method comprising the steps of: determining whether the patient has the COVID-19 by obtaining a biological sample from the patient; determining whether the patient has elevated levels of aldosterone; and, if the patient has the COVID-19 and the elevated levels of aldosterone, administering the compound.

In yet another embodiment, the present disclosure provides for a use of a compound that targets mineralocorticoid receptors to treat coronavirus disease 2019 (COVID-19), wherein the compound is used when a patient has elevated levels of aldosterone.

### DETAILED DESCRIPTION

The Virus

SARS-CoV-2 is a novel zoonotic coronavirus that emerged in Wuhan, China in November 2019 and causes the novel Coronavirus Disease 2019 (COVID-19).

Coronaviruses are part of the large Nidovirales order of viruses that all have an envelope surrounding their large positive sense single-stranded RNA coding sequence. Coronavirus genes code for three main protein groups; structural, replicating and defense, and accessory proteins. The structural proteins make up the classic spherical envelope with its club-shaped spike projections that are used to bind to target cell proteins in the first step to gaining entry to the cell.

SARS-CoV-2 uses its trimeric Spike protein and two protease proteins in its host to gain entry to its target cell. The first protease, transmembrane protease serine subfamily member 2 (TMPRSS2), primes the virus by cleaving the Spike protein and exposing the Receptor Binding Domain (RBD) which binds to ACE2. ACE2 is itself a protease and, as a result of binding to the RBD, cleaves the Spike protein at its second cleavage plane. The second cleavage enables the Spike protein to bind to the cell membrane and cause it to wrap around the virus and invaginate, taking the virus into the cell. TMPRSS2 is thought to cleave ACE2 just prior to invagination, essentially destroying the ACE2 protein.

SARS-CoV-2 uses ACE2 to gain entry to cells, destroying the protein in the entry process. Viral cycles continue until the ACE2 proteins are depleted or the host immune response is activated. Higher levels of ACE2 expression will increase viral tropism and replication. This may explain why children and healthy adults, without RAS stress and the subsequent high ACE2 levels, are unlikely to suffer with this infection.

In this way, cells and tissues with higher ACE2 expression enable increased viral tropism and repeated viral cycles through the cell, vastly increasing viral replication and viral load in the host. Equally, viral infection causes an acute and complete depletion of membrane ACE2 in the target cells.

The Host

COVID-19 is increasingly showing that there is a consistent demographic of patients that are susceptible to infection as well as a consistent but widespread set of effects in susceptible patients. A consistent, but as yet confusing, pattern of susceptibility to mortality, including older age (>50), obesity, hypertension and diabetes warrants understanding in order to mitigate risk to these patients.

The RAS is a highly conserved system tasked with maintaining interstitial fluid homeostasis by increasing salt and water retention and blood flow. Liver angiotensinogen (AGT) is a large protein produced by the liver that is the primary signal in the RAS cascade. The renal protease, renin, cleaves AGT to form AngI. Systemic AngI is subsequently transformed on site by cell membrane proteases expressed by RAS-targeted respiratory, intestinal, renal and cardiovascular tissue.

AngI is cleaved by the protease ACE to form AngII, the final angiotensin receptor (AT₁R) agonist. AngII critically regulates blood volume, blood pressure and blood pH. It has a half-life of 30 seconds and is degraded by aminopeptidase A enzymes on red blood cells.

AngII-activated AT₁Rs in non-adrenal tissue results in cascades of intracellular events that are initiated by the increased production of reactive oxygen species (ROS), through NADPH activation, and inflammatory stress through increased Interleukin-6 (IL-6) production. Excessive AngII-mediated signaling results in cell stress (oxidative and inflammatory) and contributes to cardiovascular diseases such as hypertension and coronary artery disease.

ACE2, co-expressed on the cell membrane with ACE, essentially exerts a protective effect on RAS-targeted cells by cleaving both Ang I and AngII to form Ang (1-9) and Ang (1-7) respectively, diverting Ang II from AT₁Rs to target the anti-stress Mas receptor instead. In this simplified way, ACE2 both down-regulates Ang II stress and converts it into an anti-stress effect.

Thus, ACE2 expression is increased in RAS-targeted tissues that are considered to be under RAS stress, from excessive AngII exposure. Increased ACE2 expression and the resultant increased viral tropism resulting in ACE2 depletion combine to produce oxidative and inflammatory stress that can be lethal, to both cells and tissues and the whole person.

Individuals with highly activated RASs are thus at increased risk from this pandemic infection. Understanding the source of increased RAS activity might provide clues to the susceptibility pattern.

Poorly perfused adipose tissue in obese patients also secretes AGT. Higher AGT levels translate into increased AngII levels and the resulting increases in salt and water retention and increased blood pressure. If the blood pressure is treated and adipose tissue receives adequate blood flow, the AGT levels could be expected to increase, with the target cells being subjected to harmful chronic stress.

As it happens, obese patients with hypertension increase ACE2 expression in an attempt to reduce this chronic RAS stress. Adipose hypo-perfusion is likely exacerbated by anti-hypertensive medications. ACE inhibitors and AT₁R blockers both increase the expression of ACE2, with beneficial effects related to decreasing RAS stress. Chronically elevated RAS signaling results in a disease state characterized by cardio-metabolic syndrome and is proving to be the main risk for COVID-19 mortality.

It is hypothesized in this application that patients at risk for COVID-19 morbidity and mortality have elevated levels of aldosterone and can be identified and treated before they sicken and die. Such elevated aldosterone levels are considered to be above 30 ng/dL or 0.83 nmol/L.

COVID-19

The COVID-19 cardio-respiratory syndrome is an acute ACE2-depletion state concurrent with acute respiratory distress syndrome (ARDS) and hypoxia, setting up a lethal feed-forward loop of increasing hypoxia-induced RAS stress which compounds inflammatory stress. Excessive Ang II-mediated myocarditis with acute myocardial edema could account for the left ventricular diastolic impairment, hypotension, arrhythmia and sudden cardiac death.

There is debate whether chronic AT₁R blockers or, in direct contrast, Ang II infusion during the pulmonary crisis would be helpful in treating this disease. Although chronic AT₁R-blocker use does increases ACE2 expression, which does reduce cardio-pulmonary complications in acute and chronic inflammatory stress, in the setting of inflammation compounding acute CoV2-induced ACE2 depletion, higher pre-infection ACE2 levels would be detrimental by aiding viral tropism and increasing viral load.

AngII is currently approved for use in Germany in a clinical trial in COVID-19 patients with ARDS and shock. Although the study proposes that exogenous AngII administration will counteract hypotension frequently encountered during the inflammatory crisis, hypotension in this setting is likely myocardial in origin and could be exacerbated by AngII-mediated inflammatory stress in the myocardium.

Since COVID-19 is an acute ACE2-deficiency syndrome mediated by excessive AngII, it is not unreasonable to expect Ang II administration to worsen the condition. If anything, AT₁R antagonists may help during the crisis phase of the illness by blocking the elevated AngII effects caused by hypoxia and ACE2 depletion.

To date, ACE inhibiting drugs used to reduce AngII levels and AT₁R blockers to reduce the effect of AngII have not been effective enough to prevent the excessive mortality currently been encountered with COVID-19. There is a great need for a more effective treatment.

The aim of this invention is to tackle the problem downstream of the obvious heightened AngII/AT₁R activity. Although blocking the RAS cascade has proved ineffective, blocking the secondary effector loop, aldosterone, may be helpful, based on the following rationale.

Obese patients have increased aldosterone production, with a direct relationship to the degree of obesity. COVID-19 mortality is also directly related to degree of obesity. Metabolic syndrome with diabetes and renal involvement heightens the risk of death.

Aldosterone is linked to cardio-renal metabolic syndrome in a causal manner through aldosterone ability to promote arterial hypertension, dyslipidemia, glucose intolerance, arterial disease and pro-inflammatory and pro-thrombotic states.

Aldosterone is produced in the zona glomerulosa of the adrenal cortex in response to stimulation from potassium levels, adrenocorticotropic hormone and AngII. The AngII effect is again mediated by the same AT₁R with ineffective down regulating effect on aldosterone production by AT1r blockers.

Fat cell-derived protein, Complement-C1q TNF-related protein 1 (CTRP1) also increases aldosterone production both increasing the effectiveness of AngII/AT1R binding and by inducing expression of the enzyme aldosterone synthase through activation of the CYP11B2 gene. Although potassium and AngII are considered the most important stimuli of aldosterone production in healthy people, this may not hold in obese individuals where CTRP1 levels are raised and significantly enhance the AngII/AT₁R effect in the adrenal cortex.

The aldosterone effect is mediated by the mineralocorticoid receptor (MR), a member of the steroid receptor family. Aldosterone/MR biding causes the dimerized complex to translocate to the nucleus where it induces expression of genes that regulate salt and water and other mediators of cell stress.

MR-expressing cells are found in vascular endothelium heart muscle, brain, lung renal and immune cells, linking aldosterone to endothelial dysfunction (hypertension and atherosclerotic disease), cardiac disease (hypertrophy), as well as inflammation and pro-inflammatory cytokine production. The effect on fat cells has been shown to increase expression of inflammatory markers such as 11-6 and tumor necrosis factor-a (TNF-a).

Aldosterone binding to MR also mediates rapid non-genomic effects through cascades of cell protein signaling systems such as epidermal growth factor receptor (EGRF) and mitogen-activated protein kinase (MAPK).

Aldosterone also exerts epigenetic effects on the AGT-expressing genes in fat cells, effectively converting the phenotype of AGT expression to an active state in visceral adipose cells. Another epigenetic effect may relate to the CYP11B2 gene in the zona glomerulosa related to aldosterone synthase production. In this way, individuals with abdominal obesity enhance the RAS signal by enhanced AGT production in response to stimulation and enhance aldosterone production in the adrenal gland.

In this heightened aldosterone found in obesity, ACE2-derived AngI-7 may have a down regulating effect as well as the intrinsic AngII depleting effect of Ace2. However, even when ACE inhibiting and ATTR drugs are used, aldosterone levels remain elevated in obese subjects.

At extreme levels, aldosterone causes a severe inflammatory effect that results in ectopic expression of 11BHSD-2 gene and fibrosis. This is found in aldosterone target tissues such as the heart, lungs, kidney and vascular tissue.

The MR provides a potential target as a central node in the crisis phase of COVID-19, when the combination of rising I1-6 and other inflammatory cytokines and depleted ACE2 culminates in rising aldosterone levels that promotes further RAS stress, inflammatory stress and oxidative stress, to the point of death.

Antioxidants and RAS blocking drugs are proving ineffective. MR blocking drugs may be effective on their own, or add the critical mass effect to these drug regimens to save the patients life.

Eplerenone and spironolactone are MR antagonists, both currently approved by the FDA and in widespread clinical use today. Spironolactone is less selective for the MR, and, as such binds to other steroid receptors with well-recognized pro-progesterone and anti-androgen adverse effects. Eplerenone is more selective for the MR and has 500-fold lower affinity for the androgen and progestin receptors leading to fewer side effects. Other non-steroidal MR inhibitors include finerenone, apararenone and esaxerenone and would be effective in this situation.

Spironolactone could be used in doses of 25-50 mg/day, but doses up to 200 mg/day may be required in the acute situation of COVID-19. Eplerenone could be used at 25 mg twice a day, up to 200 mg twice a day.

While excess aldosterone causes sodium and water retention with potassium and magnesium depletion, blocking aldosterone effects may cause increased potassium levels, which need to be monitored. COVID-19 is known to be associated with prolonged Qt interval on ECG and torsade de pointes, both reflective of low magnesium levels. MR blocking treatment is expected to reduce the risk of cardiac adverse events.

In this invention, patients identified with COVID-19 who were showing signs of illness and had elevated aldosterone levels would be treated with MR blocking drugs. Drugs such as eplerenone and spironolactone will be used acutely during the COVID syndrome crisis to block the effect of aldosterone as well as block the RAS enhancing effects of MR stimulation that may account for treatment failure associated with known RAS inhibitors.

Treatment will also be used to prevent the known cardiac and lung fibrosis that is found to occur commonly in survivors.

Due to the spectacular medical crisis facing the whole world, it is part of this invention to use known and safe drugs, that are readily available, to treat patients facing death from COVID-19. There is no benefit at this time to seek out novel molecules that may block the MR, or reduce CTRP1 effects, as many millions of patients will have already died.

This treatment is designed to be implemented immediately and will reduce mortality significantly, saving thousands of lives, until an effective vaccine can be produced. Both drugs will be used at their known safe dosing levels, but may be required to be used at higher levels for a short duration to prevent death.

1. A use of a compound that targets mineralocorticoid receptors to treat coronavirus disease 2019 (COVID-19).

2. The use of claim 1, wherein the compound is a steroidal mineralocorticoid receptor antagonist.

3. The use of claim 2 wherein the compound is selected from the group consisting of: eplerenone and spironolactone.

4. The use of claim 1 wherein the compound is a non-steroidal mineralocorticoid receptor inhibitor taken from the group consisting of: finerenone, apararenone and esaxerenone.

5. The use of claim 3 wherein the compound eplerenone is administered in a range between 25-200 mg twice per day.

6. The use of claim 3 wherein the compound spironolactone is administered in a range between 25-200 mg per day.

7. A method for treating a patient with a compound that targets mineralocorticoid receptors, wherein the patient is suffering from coronavirus disease 2019 (COVID-19), the method comprising the steps of:
determining whether the patient has the COVID-19 by obtaining a biological sample from the patient; and,
if the patient has the COVID-19, administering the compound.

8. The method of claim 7 wherein the compound is selected from the group consisting of: eplerenone and spironolactone.

9. The method of claim 8 further comprising:
determining whether the patient has an acute condition of COVID-19; and,
administering the compound spironolactone to the patient in an amount of 25-50 mg per day if the patient does not have the acute condition of COVID-19; and,
administering the compound spironolactone to the patient in an amount of up to 200 mg per day if the patient has the acute condition of COVID-19.

10. The method of claim 8 further comprising:
administering the compound eplerenone to the patient in an amount of 25-200 mg twice per day.

11. A use of a compound to prevent post-coronavirus 2019 (COVID-19) heart and lung fibrosis.

12. The use of claim 11 wherein the compound is a mineralocorticoid receptor blocking drug.

13. A method for treating a patient with a compound, wherein the patient is suffering from coronavirus disease 2019 (COVID-19), the method comprising the steps of:
determining whether the patient has the COVID-19 by obtaining a biological sample from the patient;
determining whether the patient has elevated levels of aldosterone; and,
if the patient has the COVID-19 and the elevated levels of aldosterone, administering the compound.

14. The method of claim 13, wherein the compound is a steroidal mineralocorticoid receptor antagonist.

15. The method of claim 14 wherein the compound is selected from the group consisting of: eplerenone and spironolactone.

16. The use of claim 13 wherein the compound is a non-steroidal mineralocorticoid receptor inhibitor taken from the group consisting of: finerenone, apararenone and esaxerenone.

17. The use of claim 1 wherein the compound is used when a patient has elevated levels of aldosterone.
